(19) United States
(12) Patent Application Publication
(10) Pub. No.: US 2019/0006049 A1
(43) Pub. Date: Jan. 3, 2019

(54) METHOD OF MODELLING BIOMARKERS

(71) Applicants: IXICO Technologies Limited, London Greater London (GB); Imperial Innovations Limited, London Greater London (GB)

(72) Inventors: Robin Mitja Benjamin Wolz, London (GB); Derek Lionel Glendon Hill, London (GB); Daniel Rueckert, London (GB); Ricardo Enrique Guerrero Moreno, London (GB)

(21) Appl. No.: 15/745,277

(22) PCT Filed: Jul. 14, 2016
(86) PCT No.: PCT/GB2016/052129

§ 371 Date: Jan. 16, 2018

(30) Foreign Application Priority Data
Jul. 17, 2015 (GB) 1512602.2

(51) Int. Cl. G16H 50/50; G16H 70/60

(52) U.S. Cl. CPC G16H 50/50; G16H 70/60

(57) ABSTRACT

The present invention provides a method of modelling the interaction between different biomarkers or other patient measurements and their progression along the trajectory of a neurodegenerative disease, the method comprising the steps of: a) Obtaining longitudinal biomarker profiles from multiple subjects; b) Storing the longitudinal biomarker profiles in a first memory; c) Using a computer system to perform temporal alignment of the longitudinal biomarker profiles; d) Identifying longitudinal biomarker parameters indicative of a subject at a target neurodegenerative disease stage; e) Applying the longitudinal biomarker parameters to each longitudinal biomarker profile and subject in the memory to define respective parametric models; f) defining a biomarker signature for each subject by combining two or more longitudinal biomarker profiles for that subject; and g) forming a global disease model by combining individual parametric models and biomarker signatures.

The present invention provides a method of modelling the interaction between different biomarkers or other patient measurements and their progression along the trajectory of a neurodegenerative disease.

### BACKGROUND

Alzheimer's disease (AD) had a world wide prevalence of around 26.6 million reported cases by 2006, with predictions suggesting an increase to about 100 million by 2050. Postulated interventions are more likely to be effective in early stages of the disease, hence advances in early diagnosis have the potential to make a huge impact on the overall well-being of the population, the burden to caregivers, family members and health systems.

Current research into modelling AD biomarkers has led to an increased understanding of the underlying pathophysiology of AD. Recently, the view on AD diagnosis has shifted from being seen as a binary health state, where subjects either do or do not have the disease, to a more dynamic process in which clinical and pathological biomarkers gradually begin to change well before current diagnosis criteria and continue to do so over time. Therefore, modelling the longitudinal trajectory of AD related biomarkers is an important task in the evaluation of early indicators of the disease, monitoring disease progression, and validating proposed progression models. Detailed studies into early state longitudinal AD biomarker trajectory dynamics using data-driven methods have the potential to aid the effort in the development of measures that can accurately and robustly quantify AD even before its pre-symptomatic and pre-clinical stages. Previously, hypothetical and experimental models of disease progression based on AD biomarkers, such as cerebrospinal fluid (CSF) markers, imaging (anatomical and data-driven), and cognitive scores, have been proposed. Estimating the current progress along the disease trajectory has been the focus of many studies in this field. Recently, several approaches that regard the disease progression trajectory as a continuous process have been developed. Many of the proposed frameworks rely on modelling cognitive scores such as the mini mental state examination (MMSE), the Alzheimer's disease assessment scale (ADAS), the clinical dementia rating sum of boxes (CDR-SB), and Rey's audio visual learning test (RAVL) among others, as surrogate measures of disease progression. It has been proposed to use MMSE scores, in combination of an estimated duration of symptoms, to compute an initial decline rate dubbed pre-progression rate, which is then used to classify subjects as slow, intermediate and rapid progressors. Using the estimated initial progression rate Doody et al. [1] tested the predicted survival rate, performance on cognition, function and behaviour over time in the same groups (slow, intermediate and rapid progressors) of subjects. Yang et al. [2] proposed an exponential model of ADAS scores, then used this to estimate the disease-duration and thus current pathologic stage of a subject by fitting its ADAS scores to the exponential model. Similarly, Delor et al. [3] computed a disease onset time by adjusting subjects according to their CDR-SB score. Ito et al. [4] developed a mathematical model to describe the longitudinal response in ADAS of a group of subjects, where the rate of progression was found to increase with baseline severity, age, ApoE-ǫ4 genotype, and gender were identified as covariates.

As noted by Jack at al. [5], cognitive markers are especially relevant for the advanced stages of the disease, however, they are less sensitive at earlier stages. In this regard, several modelling frameworks that exploit the complementary information of several types of AD biomarkers have also been put forward. Caroli et al. [6] tested the hypothetical model of AD biomarker dynamics proposed by Jack at al. [7] on real data. Using CSF Aβ 1-42, tau, hippocampal volume, and fludeoxyglucose positron emission tomography (FDG-PET) biomarkers it was shown that, when individual values were Z-transformed and plotted against ADAS scores, a sigmoid model could be used to describe the population time course of biomarkers. Jedynak et al. [8] propose a statistical methodology using multiple AD biomarkers, which produces for each subject and each time-point in the database an Alzheimer's disease progression score. Schmidt-Richberg15 et al. [9] propose an approach to estimate typical trajectories of several biomarkers in the full course of disease, using quantile regression via vector generalized additive models (VGAM), to derive a probabilistic approach to estimate current disease progress and disease progression rate.

Mixed effect modelling offers an appealing alternative to model generating, as it permits the incorporation of unseen subject specific random effects. To this end, Ito and Hutmacher [23] propose the use of a longitudinal non-linear mixed effect models to predict the CDR-SB time-profile, which is used to estimate the median time for clinically “worsening” and determine clinical endpoints for use in therapeutic trials. Bernal-Rusiel at al. [10, 11] propose a spatiotemporal extension of linear mixed effects modelling for mass-univariate analysis of longitudinal neuroimaging data, which as the name suggests, exploits the spatial structure in imaging data. Adak et al. [12] calculated rates of cognitive decline using hierarchical mixed effects models in which each subject is assumed to have a trajectory of cognitive decline that is linear in time, and the slope of this line measures the rate of cognitive decline. Donohue et al. [13] propose to use self-modelling regression, with linear subject-level effects and a nonparametric monotone smoothing function to model long-term features, to simultaneously estimate pathological timing and long-term growth curves.

A different type of approach that models the disease progression not as a continuous process but rather as discrete stages has also been suggested by some authors. Fonteijn et al. [14] propose the use of an event-based model to determine the order in which CSF, image-based and cognitive biomarkers become abnormal in familiar AD and Huntington's disease, to subsequently assign a subject to one of several discrete disease stages. The event-based model was later reformulated and extended by Young et al. [15] for the use in sporadic AD. However, discretizing the disease progression trajectory is a strong assumption that most likely leads to an over-simplification of the problem. To this end, Jack and Holtzman [16] argue that accurate time-dependant models of AD biomarkers must be incorporated into diagnosis criteria.

Some of the drawbacks of the mentioned approaches is their reliance on modelling whole (rather heterogeneous) populations and/or that they offer no trivial way to make predictions on the future trajectory of unseen subjects. It is a well known fact that there is a great intersubject variability and therefore a model that sets to describe a whole population could provide a suboptimal fit. Modelling a subject's biomarker's behaviour using an instantiated subpopulation of similar cases might produce a more realistic subject-specific trajectory model.

Recently, there has been a great deal of interest in temporal modelling of AD biomarkers. As AD population studies develop, more and more longitudinal data has become available. Modelling the relationships in such longitudinal data remains a challenge. In modelling biomarkers from data that contains several subjects measured at several times we are presented with several choices. On one hand it could be assumed that all measurements are drawn independently from the same general population, however, measurements are grouped (i.e. longitudinally by subject) and hence not independent. Another approach could be to fit individual models to each subject, thus treating some subject measurements as dependant while independent from the rest, this however ignores the fact that subjects themselves belong to a population.

The present invention seeks to use mixed modelling to address the aforementioned problems.

### SUMMARY

An aspect of the present invention provides a method of modelling the interaction between different biomarkers or other patient measurements and their progression along the trajectory of a neurodegenerative disease, the method comprising the steps of: a) obtaining longitudinal biomarker profiles from multiple subjects by measuring one or more predetermined biomarkers over time; b) storing said longitudinal biomarker profiles in a first memory; c) using a computer system to perform temporal alignment of said longitudinal biomarker profiles; d) defining a parametric model for each of said one or more biomarkers; e) fitting an appropriate parametric model to each longitudinal biomarker profile for each subject; g) defining a biomarker signature for each longitudinal biomarker profile; and h) forming a global disease model by combining individual parametric models and biomarker signatures.

Mixed effect modelling is used to reconcile longitudinally grouped data in a multi subject model and data contained in subject specific models. Most AD biomarkers can be expressed as models that combine both fixed and random effects, where an effect refers to anything that might have an influence on the response variable. In mixed effects models these influencing factors are considered variables that form part of the model parameters. Fixed effects are assumed to represent those parameters that are the same for the whole population and amount to traditional regression modelling. Random effects on the other hand, are group (subject) dependant random variables representing additional hidden effects, which are modelled as additional error terms with a specific distribution and covariance.

In one embodiment a Local Model is instantiated from the Global Model of step (g) to describe the trajectory of a test subject with unknown status, the method comprising the further steps of: h) extracting the biomarker signature of a test subject from the first memory; i) using the computer system to evaluate the similarities between the biomarker signature of the test subject and the biomarker signatures of all subjects in a database; j) selecting for each longitudinal biomarker profile the individual parametric models that are most relevant to the test subject according to biomarker signature similarity; k) assigning weights to one or more model parameters of each parametric model; and l) building a local disease model for the test subject based on the model parameters defined in j) and k).

### FIGURES

Certain embodiments of the invention will now be described by way of reference to the following figures:

FIG. 1 shows an illustrated diagram of a method according to embodiments of the invention;

FIG. 2(a) illustrates an example of model estimation from nearest neighbours based on biomarker signatures;

FIG. 2(b) illustrates an example of model estimation from nearest neighbours based on biomarker change over time;

FIG. 3 illustrates an example of a quantile model matching and shift estimation process;

FIG. 4 illustrates a longitudinal distribution of a subset of data relating to a complete set of biomarkers associated with Alzheimer's Disease;

FIG. 5 illustrates time adjusted biomarker score values of CN-MCI converters, MCI-AD converters and CN-MCI-AD converters aligned using the quantile matching and shift estimation process illustrated in FIG. 3.

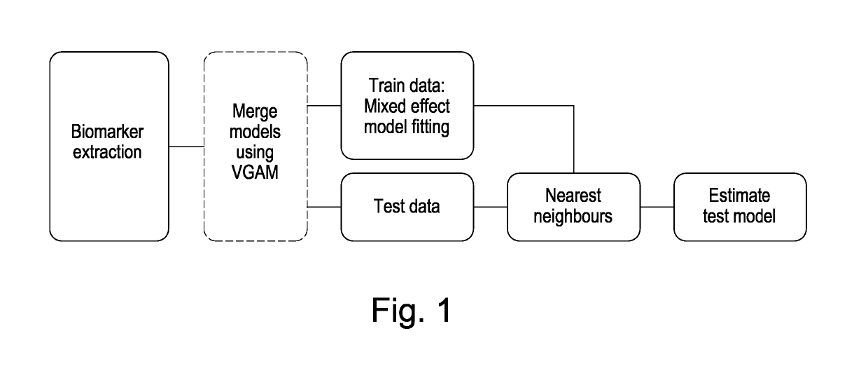

### DESCRIPTION

The invention will now be described with reference to the certain embodiments of the invention.

A mixed effect model relating to the j^{th} observation of an i^{th} individual can be written as:

yᵢⱼ=ƒ(Φᵢ, Xᵢⱼ)+εᵢⱼ, (1)

where yᵢⱼ is the j^{th} response of the i^{th} subject, xᵢⱼ is the predictor vector for the j^{th} response on the i^{th} subject, f is a function of the predictor vector and a parameter vector Φᵢ of length r, and εᵢⱼ is a normally distributed noise term. As mentioned before, mixed effects models contain both fixed (population) and random (individual) effects. Hence, the parameter vector Φᵢ can be split in and written as:

Φᵢ=Aᵢβ+Bᵢbᵢ bᵢ˜N(0, σ²D), (2)

where β is a p-vector that represents the parameters corresponding to the fixed effects, bᵢ is the i^{th} subject's q-vector of random effects, Aᵢ and Bᵢ are r×p and r×q design matrices, and σ²D is the covariance matrix. If design matrices Aⁱ and Bⁱ are identity matrices l, this implies that all model parameters have associated random effects and that the same fixed effects apply to the whole population. A more sophisticated choice of design matrices Aⁱ and Bⁱ can be used to add covariates to the model, such as age, ApoE-ε4 genotype or education. Equation 1 can represents either linear or nonlinear mixed effect models by changing the parametric modelling function ƒ(Φ, x), which should be determined by the underling process being modelled.

### Types of Parametric Models

Theoretical long-term physiological and psychological biomarker models been hypothesized to follow a sigmoid shape. Furthermore, experimental evidence also suggests that linear models do not sufficiently portray cognitive or functional decline in disease progression. The use of sigmoidal curves to describe non-linearity in cognitive and functional decline is well accepted. Additionally, sigmoidal models also offer the advantage of fixing the lower and upper model asymptotes to the scale's limits, so that model predictions do not fall outside this bounded scale.

On the other hand, some evidence also suggests a sigmoidal pattern, with an acceleration phase during the early stages of the disease and deceleration at later stages, for the dynamic behaviour of cortical thinning and hippocampal volume loss. However, in little evidence of acceleration, that would suggest a non-linear effect, in structural brain atrophy rates was observed. Therefore, an embodiment of the present invention simplifies structural MR volumetric biomarker dynamics by using a linear function.

In principle, the dynamic behaviour of machine learning derived biomarkers is less well understood, and generally heavily depends on the algorithm and parameter choices used to derive them. Two types of biomarkers fall into this category: manifold based and grading features. Both of these type of biomarker parametric model choices were made empirically, were sigmoid and quadratic models where used for manifold based features, while sigmoid models where used for grading features.

Model choices for all tested biomarkers were empirically justified by fitting several other non-linear, or in some cases linear models. In total, three different parametric functions were used depending on the biomarker being modelled, mainly, sigmoid, quadratic and linear:

$$f ( φ , x ) = \frac{{φ}_{1}}{1 + {e}^{- {φ}_{2} ( x - {φ}_{3} )}} + {φ}_{4} \\ f ( φ , x ) = {φ}_{1} + {φ}_{2} x + {φ}_{3} {x}^{2} \\ f ( φ , x ) = {φ}_{1} + {φ}_{2} x . \qquad ( 3 )$$

### Model Averaging

Generally, several individual curves of the same type and their arithmetic mean do not necessarily maintain the same behaviour. An objective of an embodiment of the invention is to build models based on averaging several individual curves. However, the arithmetic mean needs not to display the same characteristics as the individual curves. That is, the arithmetic mean of several sigmoid functions does not follow a sigmoid shape. Here, the assumption is that individual models should behave in the same way as the mean population model, hence the assumption that averaging several individual model curves should give result to a mean model curve of the same type is imposed, e.g. averaging sigmoid curves should produce a sigmoid curve in order to fit with the hypothesis model. Mathematically, this assumption can be validated as follows. Suppose that a biomarker is modelled by an arbitrary function:

y=ƒ(Φ,x)+ε (4)

where y represents the biomarkers evolution, x represents time, and Φ are the arbitrary constants of the model. Then, individuals 1, 2, . . . , n belonging to this population can be modelled as:

$$\begin{matrix}{y}_{1} = f ( {φ}_{1} , x ) \\ {y}_{2} = f ( {φ}_{2} , x ) \\ ⋮ \\ {y}_{n} = f ( {φ}_{n} , x )\end{matrix} \qquad ( 5 )$$

If [symbol] is considered to represent the mean biomarker value at time x, then:

$$\overline{y} = \frac{1}{n} [ f ( {φ}_{1} , x ) + f ( {φ}_{2} , x ) + … + f ( {φ}_{n} , x ) ] . \qquad ( 6 )$$

Constants of individual curves can be expressed as deviations from the mean model in an analogues way as the random effects in a mixed effects model, and thus can be written as:

ϕ=ϕ̅+ψ, (7)

Where ϕ̅ are the constants' values of the mean model and ψ are the constants' deviation from the mean, and an individual's model can be written as:

ƒ(ϕ,x)=ƒ(ϕ̅+ψ,x). (8)

Expanding Equation (8) by Taylor's series,

$$f ( \overline{φ} + ψ , x ) = f ( \overline{φ} , x ) + ψ {f}_{φ}^{′} + \frac{1}{2 !} ( {ψ}^{2} {f}_{φ}^{″} ) + … \qquad ( 9 )$$

Where

$${f}_{φ}^{n} = \frac{{\partial }^{n} f}{\partial {φ}^{n}} .$$

Any new individual's model can be estimated and expressed as a series expansion, the first term of which is a function of the same form as all the other curves that are involved in describing it. Additionally, the arbitrary constants of the first term are the constants' means of the separate curves. The remaining terms of the series express the divergence from the curve

[symbol]

=ƒ(ϕ̅,x).

The mean n of such curves is,

$$\overline{y} = f ( \overline{φ} , x ) + {f}_{φ}^{′} \frac{Σψ}{n} + \frac{1}{2 !} ( {f}_{φ}^{″} \frac{{Σψ}^{2}}{n} ) + … \qquad ( 10 )$$

The underlying assumption is that all individuals' trajectories (of a specific biomarker) are represented by the same model (albeit with different parameters). Therefore, only the first term of Equation 10 is considered here, and averaging several curves reduces to averaging their parameters Φ.

### Out-of-Sample Model Estimation

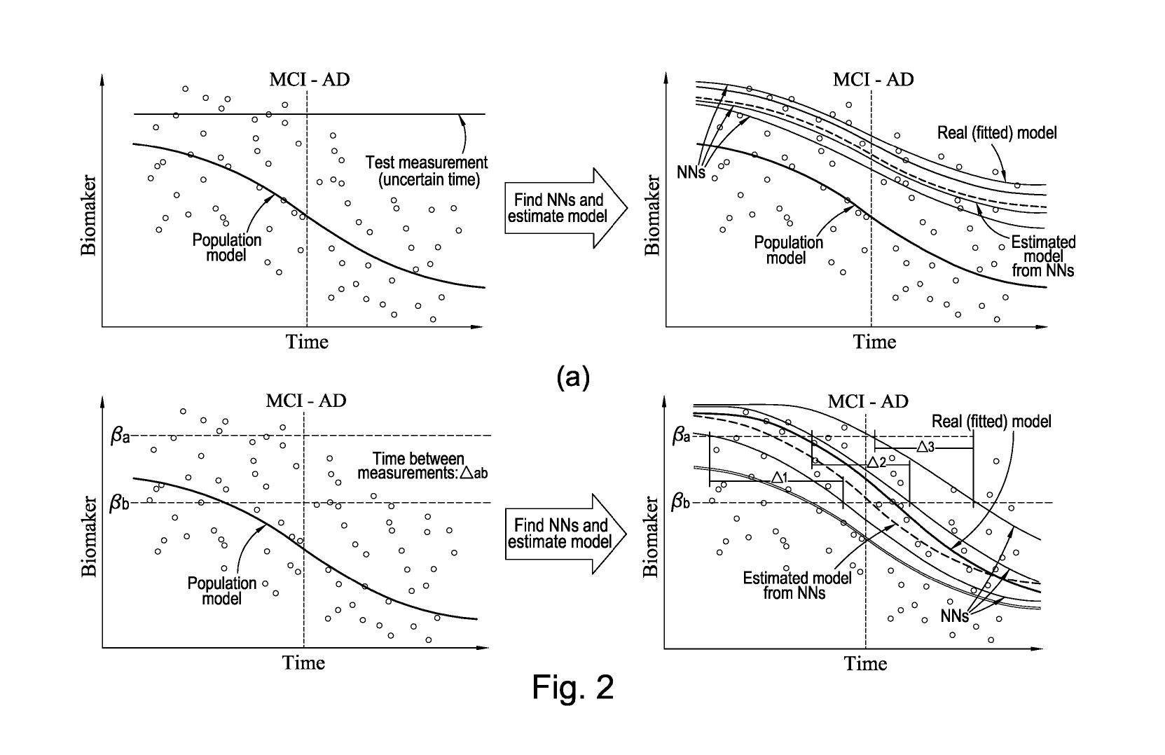

Using mixed effect modelling population, individual models can be obtained for a training dataset of subjects and their respective biomarkers. However, given an unseen subject with one (or more) time points and at least one measured biomarker the objective is to estimate its current “state” and predict its future behaviour along the disease trajectory, that is, estimating the current time-to-conversion and modelling the progression path as can be seen in FIGS. 2(a) and 2(b). In FIG. 2(b) a biomarker β is measured at time points a and b, where the time difference between measurements is Δ_{ab}. Nearest neighbours are selected as those training models that given biomarkers βₐ and β_{b}, produce a measurement time difference Δᵢ similar to Δ_{ab}.

Some biomarkers might be associated with different aspects of the disease, or rather with different aspects of how the disease is modelled. Choosing the right set of biomarkers, the relevant “biomarker signature”, to determine subject similarity and hence define an instantiated subpopulation of similar cases for parameter estimation, can therefore play an important role. Here, the correlation coefficient between all training subject's model parameters and all biomarkers is used to find those biomarkers that are correlated with the change in specific biomarker model parameters. At training time, these coefficients are used to choose the top (available) biomarkers to compose a biomarker signature which in turn is used to measure similarity between a test subject and the training set and find the most similar cases for each parameter independently. FIG. 2 (a) illustrates the process of estimating a progression model for an unseen subject for one biomarker using its biomarker signature. In principle, the obtained biomarker signature might be a good way to approximate the current disease “state”, however, this is not necessarily the case for disease progression speed estimation (e.g. the slope in a linear model). If more than one time point is available, test subject instantiation can also take into account biomarker change over time between visits. FIG. 2 (b) illustrates the process of the subpopulation instantiation based on biomarker change. Both this measures (biomarker signature and speed) are normalized and combined to find a test subjects nearest neighbours and estimate its biomarker's progression models.

### Model Merging

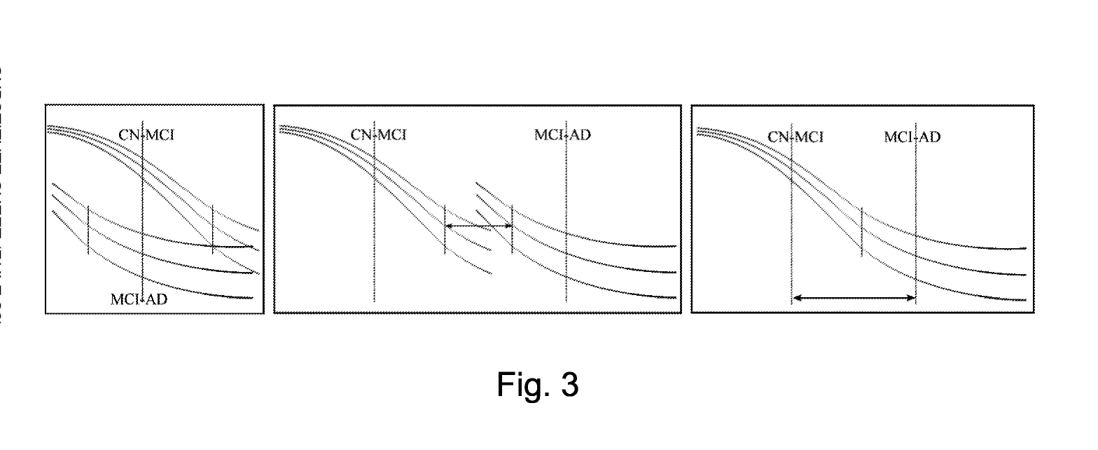

The proposed framework relies on the temporal alignment of features based on the conversion to a more severe disease label. However, due to the temporal limitations of most population studies, it is often difficult to observe subjects going through all the phases of the disease trajectory, that is, a subject starting the study as a healthy control (CN) subjects, moving to a high-risk or mild cognitive impairment (MCI) phase and finally being diagnosed as probable AD. It is more often the case that subjects progress from CN to MCI or from MCI to AD, with a different model needed to explain each of these two phases due to the lack of a consistent temporal alignment variable. With this in mind, an extended disease trajectory model can be learned by merging the CN-MCI and MCIAD models. This can be achieved using the cross-sectional variance similarity of both models in an overlapping temporal region. This overlapping region is assume to be before MCI subjects converts to AD and after CN subjects have converted to MCI. To this end, the LMS (lamda-mu-sigma) method in combination with VGAM [41] can be used in the same way to calculate a quantile regression fitting for each section of the model (CN-MCI and MCIAD). The conditional distribution (or quantile positioning) of the biomarker at each time (after temporal alignment) are used as a measure of model similarity, and the maximization of such inter-model similarity determines the temporal shift between models. The search space for the model shift is constrained to be somewhere between CN-MCI and MCI-AD conversion. FIG. 3 illustrates the quantile matching and model merger process.

### LMS and Vector Generalized Additive Models

The LMS method is a popular technique for quantile regression due to its flexibility and simplicity. The main idea behind the LMS method is the maximization of a penalized log-likelihood of the three parameters of a Box-Cox power transformation (the Box-Cox power λ, the mean μ and the standard deviation σ) of the dependant variable y (a biomarker measurement here), such that y approaches normality. The transformation not only enforces normality but also completely summarize its distribution over the range of the covariate x (time to/from conversion here). The desired quantile curves are then back-transformed to the original (and most likely not normally distributed) space through the inverse Box-Cox power transformation.

A significant drawback of the LMS method relates directly to the Box-Cox power transform, where the dependant variable is required to have positive values. Quantile regression using VGAMs provides a larger unified theory and estimation process. VGAMs are a non-parametric extension of vector generalized linear models (VGLMs) that estimate the conditional distribution ƒ_{y}(y|x), where ƒ_{y} is assumed to be given by a smooth function g_{y}(y, η1(x), . . . ηN(x)). Here, nn are the predictors given by ηₙ(p)=βₘ(p)+ƒₙ(p), βₙ are intercept values and ƒm can be estimated with a running lines, kernel or spline smoother [20]. The Yeo-Johnson power transformation [42] can be incorporated to handle non-positive values of y as the same time as enforcing normality, and can be written as:

$$ψ ( λ , y ) = \{ \begin{matrix}\{ {( y + 1 )}^{λ} - 1 \} / λ & ( y \geq 0 , λ \neq 0 ) \\ log ( y + 1 ) & ( y \geq 0 , λ = 0 ) \\ - \{ {( - y + 1 )}^{2 - λ} - 1 \} / ( 2 - λ ) & ( y < 0 , λ \neq 2 ) \\ - log ( - y + 1 ) & ( y < 0 , λ = 2 )\end{matrix} \qquad ( 11 )$$

Additionally, the conditional distribution g_{y} at x depends on three parameters_=[λ(x), μ(x), σ(x)] and is given by:

$${g}_{y} ( y , λ ( x ) , μ ( x ) , σ ( x ) ) = \frac{1}{σ ( x )} φ ( \frac{ψ ( y , λ ( x ) ) - μ ( x )}{σ ( x )} ) {( \vert y \vert + 1 )}^{sgn ( y ) ( λ ( x ) ) - 1 )} , \qquad ( 12 )$$

where ϕ(·) denotes a standard normal distribution. The obtained conditional distributions of the CN-MCI and MCI-AD models can be matched to find the optimal offset between the two models. Conditional distribution overlap (area under the curve) is used as a matching criteria. An offset is found for each biomarker's two models and a weighted average can be obtained using the area under the curve of the conditional probabilities as weighting factor. Once the offset is found, the mixed-effect-models can be trained using the offsetted sample times, to obtain a complete CN-MCI-AD model.

### Data

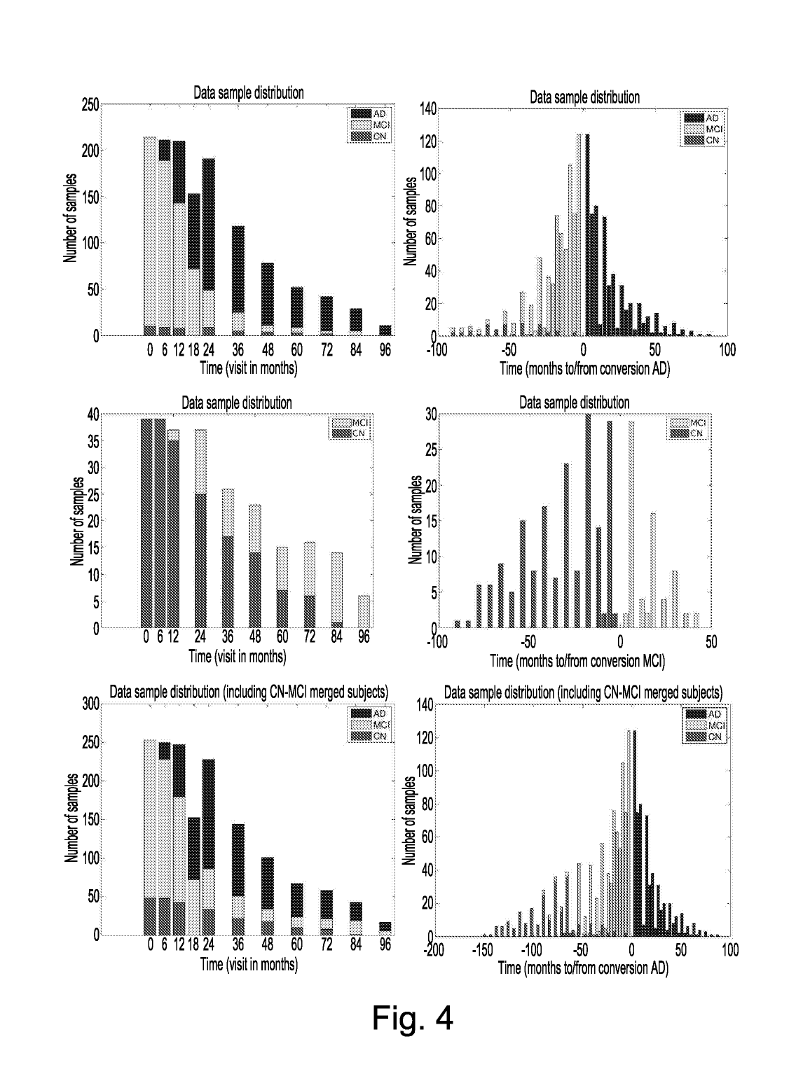

Data used in the preparation of this work was obtained from the Alzheimer's Disease Neuroimaging Initiative (ADNI) database. ADNI is a large-scale multisite study that aims at analysing biomarkers from cognitive tests, blood tests, tests of cerebrospinal fluid, and MRI/PET imaging with regard to their ability to characterise the progression of AD. To date, ADNI in its three studies (ADNI-1, -GO and -2) has recruited over 1500 adults, aged between 55 and 90 years, to participate in the study. Participants consist of cognitive normal subjects (CN), subjects with significant memory concerns (SMC), MCI or early MCI (eMCI), and early AD. Here, all subjects enrolled in either ADNI-1, ADNI-GO or ADNI-2 are considered (based on the ADNIMERGE data base as of 19 Feb. 2015). Of particular interest in this work are the subset of subjects that have at least four different time point samples and that convert to AD at some point during study. Different amount of samples of each subject are available for the different biomarkers considered here, e.g. not all available time points of a subject might contain some cognitive or imaging features. 78 subjects that reverted to a less severe stage in the disease at some point during the study were excluded. In total, there were 1153 subjects with at least four time points of any biomarker (6407 individual entries). Out of these, 216 subjects (1309 samples) converted to AD (includes 10 subjects that start as CN), 39 subjects (with 252 samples) converted from CN to MCI (excludes subjects that start as CN but convert to AD) and had available all biomarkers at all time points. Evaluations are either done with the 216 subject set of MCI-AD or the set of 255 CN-MCI-AD converters that contain all biomarkers in order aid in comparisons. As can be expected in a large longitudinal study, data distribution is not longitudinally uniform due to subjects dropping out of the study, as can be seen in FIG. 4 for the MCI-AD and CN-MCI groups on the top two rows, respectively. Additionally, FIG. 4 bottom row shows the distribution of the data once the CN-MCI data has been time shifted to the AD-converter domain (see Section 4 for details). Out of the 1561 available samples 974 were pre-AD and 587 post-AD diagnosis, that is, there are %25 more pre-AD samples. Biomarkers considered in this work can be divided into two broad categories, cognitive and imaging, with the latter further subdivided into volumetric, manifold learning and grading.

### Cognitive Features

ADNI participating subjects were asked to perform a battery of cognitive test at each visit during the study. The direct total score of each of these cognitive tests was used here as a biomarker. Cognitive tests included in this work include MMSE, ADAS11, ADAS13, the Functional Activities Questionnaire (FAQ), CDR-SB and RAVLT. As noted before, not all results were available at every visit due to unspecified causes. Thus, the number of available subjects for the mixed-effect-model fitting varied from 48-49 (with 332-339 samples) for the CN to MCI conversion model and from 264-269 (with 1697-1740 samples) for the conversion to AD model.

### Volumetric Features

MR images were automatically segmented into anatomical regions, for which multi-atlas label propagation with expectation-maximisation (MALPEM) described in [27] was employed. Here, 30 atlas segmentations are transformed to an un-segmented image using a robust non-rigid registration approach based on multi-level free form deformations. The individual atlas label maps are then transformed to the image space of the un-segmented image using the calculated transformation and a nearest neighbour interpolation scheme. The propagated atlas label maps are then fused into a consensus probabilistic segmentation using a local weighting approach. From this 134 probabilistic label estimates (corresponding to the 134 anatomical regions in the atlases) are obtained. Further refinement using a method that exploits image intensities in an expectation-maximisation framework is carried out. From the 134 anatomical structures 17 are selected, based on their overall temporal discriminability, for the analysis in this work. The total number of training subjects available was 41 (with 264 samples) for CN to MCI model and 224 (with 1379 samples) for conversion to AD model. This can be seen in FIG. 4.

### Manifold Based Features

Features obtained from MR images using manifold learning (ML) have been shown to contain valuable information about disease severity and progression. The aim is to learn the underlying low-dimensional manifold that best represents a high-dimensional population, such that similar scans have similar low-dimensional coordinates. Here, this is achieved in three steps: First, regions of interest (ROI) relevant to disease progression are learned using sparse regression from MR images. Second, local binary patterns (LBP) around each ROI voxel are extracted to transform MR intensities to an augmented higher-dimensional binary space. Finally, Laplacian Eigenmaps is used to learn the low-dimensional manifold. Local geometry is determined via a sum of squared differences (SSD) similarity matrix. A sparse k-neighbourhood graph of the data points is built, with the additional constraint that at most one instance per subject is admitted into the neighbourhood . The manifold is chosen to have two dimension, namely D1 and D2, with a third feature P_D1D2 obtained by combining D1 and D2 via a perpendicular projection of the points in R² to a fitted quadratic curve. Manifold features were computed for 1063 subjects (5679 images) that had at least four visits, out which 41 subjects (with 264 images) where used for CN to MCI model fitting and 224 subjects (with 1379 images) for the conversion to AD model.

### Grading Features

The goal of grading features is the scoring of a test subject's image ROI by estimating its nonlocal similarity to different training populations. There are two main steps in the calculation of the grading features: (1) A ROI is first learned using sparse regression; (2) Based on the learned ROI, the disease labels of training subjects were propagated to test subjects to calculate a disease grading score for each test subject. The relation between the training population and each test subject is modelled using elastic-net regression. Given the intensities of a test subject Xₜₑₛₜ∈R^{k×1} and of n training subjects X_{training}∈R^{k×n} (within the learned ROI), the grading score gₜₑₛₜ of the test subject can be calculated by minimizing the following cost function:

$$\{ \begin{matrix}\hat{α} = {min}_{α} \frac{1}{2} {‖ {X}_{test} - {X}_{training} α ‖}_{2}^{2} + {λ}_{1} {‖ α ‖}_{1} + \frac{{λ}_{2}}{2} {‖ α ‖}_{2}^{2} \\ {g}_{test} = \frac{{\sum }_{j = 1}^{n} {\hat{α}}_{t} ( j ) {I}_{j}}{{\sum }_{j = 1}^{n} {\hat{α}}_{t} ( j )}\end{matrix} \qquad ( 13 )$$

Here, ̂α are the coding coefficients of the test subject and lⱼ is the disease label for the jₜₕ training subject. CN and AD subjects were used for training, where if the training image belonged to a CN or AD subject, lⱼ was set to 1 or −1, respectively. In order to calculate robust grading features, we added a constraint that only one time point of a training subject can be selected for label propagation. In addition, a re-sampling scheme was employed to reduce sampling bias and enable a robust feature calculation. Finally, the calculated grading scores can be used as features for analysis.

### Model Training and Merging

Linear models were trained using Matlab's R2014a nlmefit, while non-linear models were trained using Matlab's R2014a nlmefitsa. In these functions, mixed and random effect parameters estimates that maximize a likelihood function are sought. In nlmefit the likelihood is approximated then maximized, while nlmefitsa maximizes the exact likelihood function though a random parameter estimation process. Results from the non-linear mixed effect model estimation are random and vary from run to run due to the estimation process itself. Here, parameters from 10 different runs are averaged in order to obtain the final model.

Linear models were used for volumetric biomarkers as they were observed to show an approximately linear behaviour during the study's duration. A sigmoidal function was used to model cognitive and grading biomarkers, were the lower and upper asymptotes were fixed at the limits of the cognitive tests and to −1 and 1 for the grading features, which reduced the number of free parameters in the model to two. The first dimension of the manifold D1 was also modelled as a logistic function, however both the upper and lower asymptotes were left as free parameters as there is no theoretical lower or upper bound in the manifold coordinates. Finally, D2 and P D1D2 from the manifold where modelled as quadratic functions. Other choices of models considered based on literature and observations were the exponential and Gompertz functions, but were ultimately rejected as they did not fit as well the observed data.

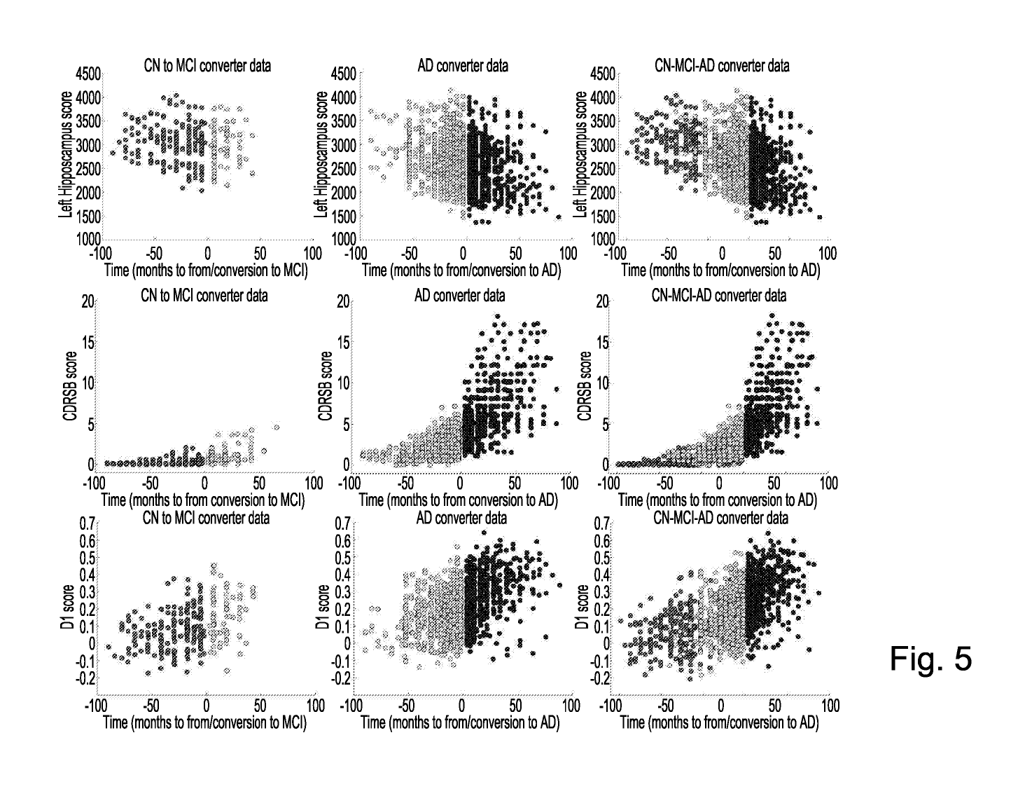

The conditional distribution (or quantile positioning) of the biomarker at each time (after temporal alignment) is used to determine the temporal shift between CN-MCI and MCI-AD models. A VGAM is fitted to each biomarker to find the conditional distributions of the CN-MCI and MCIAD conversion models. It must be noted that VGAMs could not be fitted to CDRSB and FAQ biomarker's CN-MCI conversion models, as a large portion of biomarker samples clustered at zero, and therefore were omitted from the model shift calculation. Averaging the obtained shift from the remaining biomarkers yielded a shift of around 59 months (or ˜1800 days), in other words, conversion from CN to MCI is assumed to happen 59 months before conversion from MCI to AD. Comparable results of 1860 days were found by Schmidt-Richberg [37], where the same technique but a different set of subjects and biomarkers were used. FIG. 5 shows the resulting CN-MCI, MCI-AD and CN-MCI-AD models for some biomarkers.

### Time-To-Conversion Estimation

Disease progress or state estimation is of great interest to Alzheimer's disease researchers, clinicians and caregivers. However, information about the current disease state of a subject is generally unknown and needs to be estimated. Here, time-to-conversion (TTC) is used as a surrogate measure of the current disease state. On its own TTC is not sufficient to give a current disease state as it ignores the speed at which the disease will progress in a particular subject. Nonetheless, as part of the proposed framework an individual progression model for each biomarker of each subject is obtained, which can then be compared to the population model in order to find a relative progression speed. In this regard, TTC estimation in combination with a speed factor for each test subject can give a relative estimate of the current disease state and speed. As mentioned before, the interest here lies in estimating the TTC to/from AD, and as explained above two types of model can be used:

The AD conversion model (referred as MCI-AD), that uses as training subjects that convert to AD during the study (including subjects that start as CN but convert to AD), and the CN-MCI-AD model, which merges the MCI-AD and CN-MCI (subjects that convert from CN to MCI) models. TTC is estimated by finding similar cases of a test subject in the training set, averaging the models of those similar cases to estimate the test subjects individual model, and finally using the model to estimated TTC.

There are three types of model being considered and compared to make predictions: a) the resulting population model (popMod) from the mixed-effect regression (fixed effects), b) the instantiated model based on nearest neighbours (nnMod), and c) the “real” model (realMod), which is the model fitted to each subject in the mixed-effect regression framework (fixed+random effects). Assuming that the chosen parametric function correctly describes the biomarker's behaviour, real Mod can be seen as the “real” trajectory (best case scenario) and deviations from it could be considered measurement noise. The aim is to examine if the estimated nnMod is a better approximation to the realMod than the popMod. The estimation process of the nnMod is based on the averaging of the models (from the mixed-effect modelling) of the nearest neighbours of the test subjects.

Considering that each subject has four or more time points, two possible scenarios are explored: Cross-sectional estimation, where each instance of the test subject can be treated independently to estimate its “current” disease state. Longitudinal estimation, where one of the subjects instances can be used as anchor to estimate its current and future (or past) disease states. A leave-one-out experiment was carried out where each subject was removed from the training set and treated as an unseen subject. Tables 1 and 2 (rows marked as: 1 time point) detail the results of cross-sectional and longitudinal TTC estimation. For the longitudinal TTC estimation an anchor point needs to be chosen in order to find its nearest neighbours and estimate its corresponding model. Three different anchor points where examined, mainly, baseline, last MCI and all MCI time points, to estimate a model and compare model predictions to actual TTC.

TABLE 1
Cross-sectional TTC estimation mean absolute error (standard
deviation in parenthesis) of popMod, nnMod and realMod using
MCI-AD and CN-MCI-AD models. Statistical significance
(p < 0.01) between MCI-AD and CN-MCI-AD
models indicated by *.

| Model | popMod | nnMod* | realMod |
|---|---|---|---|
| MCI-AD | 22.1 (16.8) | 13.2 (12.5) | 6.0 (6.5) |
| CN-MCI-AD | 23.1 (17.3) | 13.9 (12.4) | 6.1 (6.5) |

In an additional experiment, where more than one time point is made available at test time, longitudinal information was incorporated to the nnMod model estimation process. Here, model parameters related to the offset or current disease state are estimated based on the nearest neighbours according to its biomarker signature. However, model parameters associated to the slope or curvature of the model are estimated based on the nearest neighbours according to two criteria: biomarker and temporal goodness of fit. Biomarker fit is determined by fixing the (known) time separation Δt between test time point and finding those models in the training set that produce similar biomarker values for time points separated by the same Δt. Temporal fit is determined by fixing the (measured) biomarker separation Δb between test time point and finding those models in the training set that produce similar a similar time separation Δt for time points separated by the measured Δb. Table 2 (below) gives the results of TTC estimation given different number of time points available during testing.

TABLE 2
Longitudinal TTC estimation mean absolute error (standard deviation
in parenthesis) of popMod, nnMod and realMod using MCI-AD and
CN-MCIAD models. Statistical significance (p < 0.01) between MCI-AD
and CN-MCIAD, using baseline, last MCI and all MCI as anchor
points is indicated by ⋄*†, respectively.

| Model | Anchor point | popMod | nnMod | realMod |
|---|---|---|---|---|
| MCI-AD | Cross-sectional | 22.1 (16.8) | 13.2 (12.5) | 6.0 (6.5) |
| | Baseline (1 time point) | 22.1 (16.8) | 14.2 (12.8) | 6.0 (6.5) |
| | Baseline (2 time points) | | 13.4 (12.5) | |
| | Baseline (3 time points) | | 12.8 (12.1) | |
| | Baseline (4 time points) | | 12.2 (12.1) | |
| | Last MCI (1 time point) | 22.1 (16.8) | 10.8 (10.0) | 6.0 (6.5) |
| | Last MCI (2 time ponts) | | 10.8 (10.0) | |
| | Last MCI (3 time ponts) | | 10.8 (10.3) | |
| | Last MCI (4 time ponts) | | 18.9 (10.3) | |
| | All MCI (1 time point) | 22.1 (16.8) | 18.2 (12.8) | 6.0 (6.5) |
| | All MCI (2 time points) | | 17.7 (12.5) | |
| | All MCI (3 time points) | | 18.9 (12.5) | |
| | All MCI (4 time points) | | 18.3 (19.1) | |
| CN-MCI-AD | Cross-sectional | 23.1 (17.3) | 19.9 (12.4) | 6.1 (6.5) |
| | Baseline (1 time point) | 23.1 (17.3) | 13.7 (12.0) | 6.1 (6.5) |
| | Baseline (2 time points) | | 13.0 (11.9) | |
| | Baseline (3 time points) | | 12.1 (12.6) | |
| | Baseline (4 time points) | | 12.3 (11.8) | |
| | Last MCI (1 time point) | 23.1 (17.3) | 11.9 (10.3) | 6.1 (6.5) |
| | Last MCI (2 time ponts) | | 12.0 (10.9) | |
| | Last MCI (3 time ponts) | | 11.9 (10.8) | |
| | Last MCI (4 time ponts) | | 11.6 (10.7) | |
| | All MCI (1 time point) | 23.1 (17.3) | 15.8 (12.0) | 6.1 (6.5) |
| | All MCI (2 time points) | | 16.0 (11.9) | |
| | All MCI (3 time points) | | 16.0 (12.1) | |
| | All MCI (4 time points) | | 16.1 (11.8) | |

### Discussion

Subject visits on average occur every ˜8.7 months and we take conversion to have occurred at the mid point between the last visit with the least severe disease label and the first visit with the proceeding (more severe) label. Hence, assuming that conversion could have occurred at any point in between visits (with equal probability), it should be noted that there is on average an apriori mean absolute error of ˜2.18 (8.7/4) due to the exact conversion time uncertainty. Results presented here should be considered within this context, as this is the absolute minimum expected error due to the average biomarker sampling rate. Estimated models perform much better to the average population model in the −40 to 40 month range (close to the best case scenario of the individual fitted model). However, they perform worse outside this range (specially before), although it must be noted that there are far less data points available outside the −40 to 40 month range (see FIG. 4). Adding more time points to the model estimation process slightly improves over all accuracy. However, this generally occurs in the −40 to 40 range, while outside this range results generally degrade Sensor data at the extremes of the biomarkers (cognitive and grading) as in Ito et al. 2014.

TABLE 3
Imaging features only longitudinal TTC estimation mean
absolute error (standard deviation in parenthesis) of popMod,
nnMod and realMod using MCI-AD and CN-MCI-AD models.
Statistical significance (p < 0.01) between MCI-AD and
CN-MCI-AD, using baseline, last MCI and all MCI as anchor
points is indicated by ⋄*†, respectively.

| Model | Anchor point | popMod | nnMod⋄*† | realMod |
|---|---|---|---|---|
| MCI-AD | Baseline | 27.8 (21.2) | 16.7 (15.2) | 7.0 (7.9) |
| | Last MCI | 27.8 (21.2) | 11.9 (11.5) | 7.0 (7.9) |
| | All MCI | 27.8 (21.2) | 20.3 (15.2) | 7.0 (7.9) |
| CN-MCI-AD | Baseline | 29.0 (21.9) | 16.1 (14.4) | 7.2 (8.0) |
| | Last MCI | 29.0 (21.9) | 13.7 (12.2) | 7.2 (8.0) |
| | All MCI | 29.0 (21.9) | 17.5 (14.4) | 7.2 (8.0) |

TABLE 4
Imaging features only cross-sectional TTC estimation mean
absolute error (standard deviation in parenthesis) of popMod,
nnMod and realMod using MCI-AD and CN-MCI-AD models.
Statistical significance (p < 0.01) between MCI-AD
and CN-MCI-AD models indicated by *.

| Model | popMod | nnMod* | realMod |
|---|---|---|---|
| MCI-AD | 27.8 (21.2) | 15.4 (14.2) | 7.0 (7.9) |
| CN-MCI-AD | 29.0 (21.9) | 16.2 (14.3) | 7.2 (8.0) |

### Embodiment

A particular embodiment of the present invention utilises big data to access a data resource of multiple subjects whom are anonymised and whose biomarker data is available. Biomarker data can include imaging, results of cognitive tests, demographic information, activity information from wearable sensors and blood based markers, for example. More complex biomarkers include combination of measurements from imaging and processing using an algorithm to derive a value. The invention relies on observing changes in biomarkers over time and thus deriving a longitudinal biomarker profile for each biomarker of each subject in the database. For data accuracy at least four measurements at different time points is preferable. Longitudinal biomarker profiles are derived using an algorithm within a computer system and the derived longitudinal biomarker profiles are each stored in a memory which may be remote from the data resource of subjects or may be stored in the same location.

A key step in being able to define an accurate global disease model is the ability to undertake temporal alignment of data. Temporal alignment in the context of the invention considers how different subjects relate to each other. A simple way of undertaking temporal alignment is to group subjects by age. However, although neurodegenerative disease is more common in older subjects, grouping subjects by age may result in younger subjects exhibiting signs of neurodegenerative disease being excluding from both global and local models. Grouping subjects according to a particular biomarker profile or biomarker signature results in a more accurate grouping and limits the risk of excluding subjects whom do not meet classical criteria expected from a subject suffering from a neurodegenerative disease.

The invention can be used to derive a number of different global models. One such example is a global model of all subjects considered to be suffering from a particular neurodegenerative disease refined to those subjects whose disease has progressed to a particular stage. Once subjects have been grouped or subjected to temporal alignment it is possible to observe common parameters of subjects at a particular stage of disease progression. Such observed parameters can be applied to each longitudinal biomarker profile and/or subject to derive parametric models for each. Biomarker signatures are defined for each subject by combining two or more longitudinal biomarker profiles. The biomarker signatures for each subject are then fitted with relevant parametric models developed for each available biomarker to define a global model.

A global model of a disease parameter or stage is useful in providing an overview of a population. However, in order to provide patient specific prognosis or diagnosis, a local model relating to a specific patient is required. Such a local model can be instantiated from the global model. Once a subject has been stored in the memory, that subject's data, including biomarker signature, can be extracted. A particular subject's biomarker signature is compared across the database against biomarker signatures of other subjects. This comparison enables a medical professional or computer system to assign appropriate parametric models to the particular subject and assign weights to each parameter of the relevant parametric model(s). A predetermined number of parameters closest to target parameters are selected for use in creating a local model specific to a subject. The local model may identify current disease stage, prognosis, time to conversion, for examples. Ideally, no more than fifteen parameters would be selected for use in creating a local disease model.

The local disease model may be stored in a computer memory on a local computer, in the cloud or on a portable device.

### REFERENCES

[1] R. S. Doody, V. Pavlik, P. Massman, S. Rountree, E. Darby, and W. Chan. Predicting progression of Alzheimer's disease. Alzheimer's research & therapy, 2 (1):2, 2010.

[2] E. Yang, M. Farnum, V. Lobanov, T. Schultz, R. Verbeeck, N. Raghavan, M. N. Samtani, G. Novak, V. Narayan, and A. DiBernardo. Quantifying the pathophysiological timeline of Alzheimer's disease. Journal of Alzheimer's disease: JAD, 26 (4):745-753, 2011.

[3] I. Delor, J.-E. Charoin, R. Gieschke, S. Retout, and P. Jacqmin. Modeling Alzheimer's Disease Progression Using Disease Onset Time and Disease Trajectory Concepts Applied to CDR-SOB Scores From ADNI. CPT: pharmacometrics & systems pharmacology, 2:e78, 2013.

[4] K. Ito, B. Corrigan, Q. Zhao, J. French, R. Miller, H. Soares, E. Katz, T. Nicholas, B. Billing, R. Anziano, and T. Fullerton. Disease progression model for cognitive deterioration from Alzheimer's Disease Neuroimaging Initiative database. Alzheimer's & dementia: the journal of the Alzheimer's Association, 7 (2):151-60, 2011.

[5] C. R. Jack, D. S. Knopman, W. J. Jagust, L. M. Shaw, P. S. Aisen, M. W. Weiner, R. C. Petersen, and J. Q. Trojanowski. Hypothetical model of dynamic biomarkers of the Alzheimer's pathological cascade. The Lancet Neurology, 9 (1):119-128, 2010.

[6] A. Caroli and G. B. Frisoni. The dynamics of Alzheimer's disease biomarkers in the Alzheimer's Disease Neuroimaging Initiative cohort. Neurobiology of aging, 31 (8):1263-74,2010.

[7] C. R. Jack, D. S. Knopman, W. J. Jagust, L. M. Shaw, P. S. Aisen, M. W. Weiner, R. C. Petersen, and J. Q. Trojanowski. Hypothetical model of dynamic biomarkers of the Alzheimer's pathological cascade. The Lancet Neurology, 9 (1):119-128, 2010.

[8] B. M. Jedynak, A. Lang, B. Liu, E. Katz, Y. Zhang, B. T. Wyman, D. Raunig, C. P. Jedynak, B. Caffo, and J. L. Prince. A computational neurodegenerative disease progression score: method and results with the Alzheimer's disease Neuroimaging Initiative cohort. NeuroImage, 63 (3):1478-86, 2012.

[9] A. Schmidt-Richberg, R. Guerrero, C. Ledig, H. Molina-Abril, A. F. Frangi, D. Rueckert, and the Alzheimers Disease Neuroimaging Initiative. Multistage biomarker models for progression estimation in alzheimer's disease. In International Conference on Information Processing in Medical Imaging (IPMI), 2015.

[10] J. L. Bernal-Rusiel, D. N. Greve, M. Reuter, B. Fischl, and M. R. Sabuncu. Statistical analysis of longitudinal neuroimage data with Linear Mixed Effects models. NeuroImage, 66C:249-260, 2012.

[11] J. L. Bernal-Rusiel, M. Reuter, D. N. Greve, B. Fischl, and M. R. Sabuncu. Spatiotemporal linear mixed effects modeling for the mass-univariate analysis of longitudinal neuroimage data. NeuroImage, 81:358-370, 2013.

[12] S. Adak, K. Illouz, W. Gorman, R. Tandon, E. A. Zimmerman, R. Guariglia, M. M. Moore, and J. A. Kaye. Predicting the rate of cognitive decline in aging and early Alzheimer disease. Neurology, 63 (1):108-114, 2004.

[13] M. C. Donohue, H. Jacqmin-Gadda, M. Le Goff, R. G. Thomas, R. Raman, A. C. Gamst, L. A. Beckett, C. R. Jack, M. W. Weiner, J.-F. Dartigues, and P. S. Aisen. Estimating long-term multivariate progression from short-term data. Alzheimer's & dementia: the journal of the Alzheimer's Association, 10 (5 Suppl):S400-10, 2014.

[14] H. M. Fonteijn, M. Modat, M. J. Clarkson, J. Barnes, M. Lehmann, N. Z. Hobbs, R. I. Scahill, S. J. Tabrizi, S. Ourselin, N. C. Fox, and D. C. Alexander. An event-based model for disease progression and its application in familial Alzheimer's disease and Huntington's disease. NeuroImage, 60 (3):1880-9, 2012.

[15] A. L. Young, N. P. Oxtoby, P. Daga, D. M. Cash, N. C. Fox, S. Ourselin, J. M. Schott, and D. C. Alexander. A data-driven model of biomarker changes in sporadic Alzheimer's disease. Brain: a journal of neurology, 137 (Pt 9):2564-77, 2014.

[16] C. R. Jack and D. M. Holtzman. Biomarker modeling of Alzheimer's disease. Neuron, 80:1347-58, 2013.

1. A method of modelling the interaction between different biomarkers or other patient measurements and their progression along the trajectory of a neurodegenerative disease, the method comprising the steps of:
a) Obtaining longitudinal biomarker profiles from multiple subjects;
b) Storing said longitudinal biomarker profiles in a first memory;
c) Using a computer system to perform temporal alignment of said longitudinal biomarker profiles;
d) Identifying longitudinal biomarker parameters indicative of a subject at a target neurodegenerative disease stage;
e) Applying said longitudinal biomarker parameters to each longitudinal biomarker profile and subject in the memory to define respective parametric models;
f) defining a biomarker signature for each subject by combining two or more longitudinal biomarker profiles for that subject; and
g) forming a global disease model by combining individual parametric models and biomarker signatures.

2. The method of claim 1 wherein a Local Model is instantiated from the Global Model of step (g) to describe the trajectory of a test subject with unknown status, the method comprising the further steps of:
h. Extracting the biomarker signature of a test subject from the first memory;
i. Using the computer system to evaluate the similarities between the biomarker signature of the test subject and the biomarker signatures of all subjects in a database;
j. selecting for each longitudinal biomarker profile the individual parametric models that are most relevant to the test subject according to biomarker signature similarity;
k. assigning weights to one or more model parameters of each parametric model; and
l. building a local disease model for the test subject based on the model parameters defined in j) and k).

3. The method of claim 1, wherein each longitudinal biomarker profile comprises data taken from multiple time points.

4. The method of claim 3, wherein each longitudinal biomarker profile comprises data taken from at least four time points.

5. The method of claim 1, wherein longitudinal biomarker profiles are obtained from at least fifty subjects.

6. The method of claim 1, wherein each biomarker signature is defined by identifying one or more other longitudinal biomarker profile that are most closely related to a subject's longitudinal biomarker profile in terms of development and staging along a disease trajectory.

7. The method of claim 2, wherein the local disease model is built using the parametric models exhibiting model parameters having the highest, or lowest, fifteen weighted scores.

8. The method of claim 2, wherein the local disease model is stored in a second memory, remote from the first memory.

9. A method of predicting disease state, the method comprising:
a) obtaining one or more longitudinal biomarkers from a subject;
b) storing said one or more longitudinal biomarkers in a memory;
c) using a computer system to compare said one or more longitudinal biomarkers against a global disease model;
d) matching said one or more longitudinal biomarkers to a disease trajectory defined by the global disease model; and
e) generating a local disease model to identify disease state and/or predict disease trajectory for a subject.
